(19) United States
(12) Patent Application Publication
(10) Pub. No.: US 2008/0318308 A1
(43) Pub. Date: Dec. 25, 2008

(54) Aerating organic matter container

(76) Inventor: John Alfred Hinchey, Manchester, NH (US)

Correspondence Address: John Alfred Hinchey, 132 Glen Bloom Dr., Manchester, NH, 03109, US

(21) Appl. No.: 11/890,572

(22) Filed: Aug. 7, 2007

Related U.S. Application Data
(60) Provisional application No. 60/936,946, filed on Jun. 23, 2007.

(51) Int. Cl. C12M 1/00

(52) U.S. Cl. 435/290.1; 206/423; 220/676

(57) ABSTRACT

The present invention consists of a container of arbitrary size, shape, and density configured with ventilating holes of arbitrary size, shape, and distribution to promote air circulation and drainage. In some variations of the present invention a hinged rotatable cover or a supported removable cover prevents the entry of rain into the container. In a closed container organic material will generate dangerous heat build-up that can lead to spontaneous combustion and will present a malingering stench problem as the material decomposes. The present invention alleviates both problems by promoting and facilitating the circulation of air and the drainage of moisture while the organic material is being staged for collection and also provides additional benefits thereby inhibiting the build up of dangerous heat and objectionable fumes and reducing the possibility of spontaneous combustion. The ventilating and drainage holes significantly reduce container weight without significantly reducing strength. Furthermore, the ventilating holes and the drainage holes of the container of the present invention enable and promote evaporation and drying of the organic payload providing additional weight reduction. These additional benefits of the present invention ease the burden of lifting and transporting the waste for both the property owner and collector.

### RELATED APPLICATIONS

This application is related to and claims priority from Provisional U.S. Patent Application Ser. No. 60/936,946 filed Jun. 23, 2007, for a Aerating Organic Matter Container, with inventor John A. Hinchey which is incorporated herein by reference. The present specification focuses on general elements of the present invention. By doing so, Applicant does not intend to limit the scope of the present invention.

### FIELD OF THE INVENTION

The present invention is in the technical field of Box/Barrel/Container. More particularly, the present invention is in the technical field of containers for organic material storage and transport.

### BACKGROUND OF THE INVENTION

Many communities now include yard waste in their curbside refuse collections programs. This service is generally scheduled and provided on days designated for regular trash collection. Usually the municipality schedules the yard waste pickup weekly for a period in the spring and the fall and every other week in-between. The longer summer interval creates safety and odor problems as most residents mow the lawn at least weekly. Storing freshly mowed grass and/or brush, tree, or floral clippings in closed containers such as paper or plastic bags or traditional trash containers presents a fire hazard with the potential for spontaneous combustion and presents a malingering stench problem as the material decomposes generating heat and foul odors which are particularly onerous in the hot summer months.

In a closed container organic material will generate dangerous heat build-up that can lead to spontaneous combustion as the matter dries and decays. It is for this reason that hay must be left to dry in the field after cutting before bailing or storage in closed bays, barns, or lofts. Additionally, foul odors are generated by confined organic material.

The present invention alleviates both problems by promoting and facilitating the circulation of air while the organic material is being staged for collection. The present invention also provides additional benefits. The container is configured with multiple holes facilitating air circulation and drainage to provide natural drying, heat dispersal, decay retardation, and to impede and diffuse unsavory aromas. In some variations of the present invention, a rotatable and/or removable cover is provided to prevent the entry of rainwater while sustaining unrestricted airflow. By this method and apparatus the build up of dangerous heat and objectionable fumes is greatly diminished. The ventilating holes of the container of the present invention enable and promote evaporation and drying of the organic payload providing significant and automatic weight reduction. Furthermore, the ventilating holes themselves constitute a significant container weight reduction without significantly reducing strength. These additional benefits of the present invention ease the burden of lifting and transporting the waste for both the property owner and collector.

### SUMMARY OF THE INVENTION

The present invention is a unique container which provides and promotes the circulation of air to circumvent spontaneous combustion and to disburse foul odors while being used for the storage, transportation, and transference of organic lawn, garden, and yard matter.

The present invention is a container of variable dimensions that is configured with one or more aeration offices, zero or more drainage holes and, in some variations, a unique cover and associated fixtures for storing and transporting organic matter while suppressing attendant fire hazards and foul odors.

The container may be manifested in many variations and the configuration of aeration holes, the configuration of the drainage holes, and the configuration of the rotatable and/or removable cover and associated fixtures may assume many variations of size, shape, or pattern.

In operation the organic material is loaded into the container of the present invention and stored until transferred to the collection agency. Nothing else needs to be done by the user, the unique provisions of the container of the present invention enables and promotes air circulation thereby defusing and disbursing dangerous heat build-up and objectionable odors created by decaying organic matter.

It is an object of the present invention to provide a safe and environmentally friendly container for the storage, transport, and transfer of organic lawn, garden, and yard matter.

It is an object of the present invention to reduce or eliminate the accumulation of foul odors from the storage, transport, and transfer of organic lawn, garden, and yard matter.

Broadly stated, the present invention is a container comprising a storage medium configured with facilities to provide and promote the aeration of organic lawn, garden, and yard matter to circumvent the development of spontaneous combustion and objectionable fumes typically associated with the storage of organic matter.

### BRIEF DESCRIPTION OF THE SEVERAL VIEWS OF THE DRAWING

FIG. 1 is a front view of a representative embodiment of the present invention.

FIG. 2 is a top view of a representative embodiment of the present invention.

FIG. 3 is a perspective view of a representative embodiment of the present invention.

FIG. 4 is a front view of a first alternate representative embodiment of the present invention.

FIG. 5 is a top view of a first alternate representative embodiment of the present invention.

FIG. 6 is a perspective view of a first alternate representative embodiment of the present invention.

FIG. 7 is a front view of a second alternate representative embodiment of the present invention.

FIG. 8 is a top view of a second alternate representative embodiment of the present invention.

FIG. 9 is a bottom view of a second alternate representative embodiment of the present invention.

FIG. 10 is a perspective view of a second alternate representative embodiment of the present invention.

FIG. 11 is a front view of a third alternate representative embodiment of the present invention.

FIG. 12 is a top view of a third alternate representative embodiment of the present invention.

FIG. 13 is a front view of a fourth alternate representative embodiment of the present invention.

FIG. 14 is a top view of a fourth alternate representative embodiment of the present invention.

### DETAILED DESCRIPTION OF THE PREFERRED EMBODIMENTS

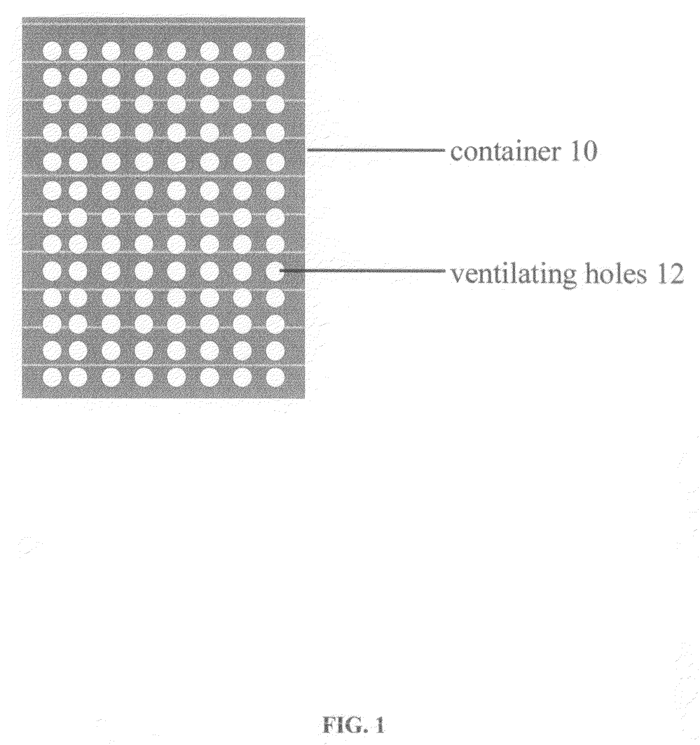

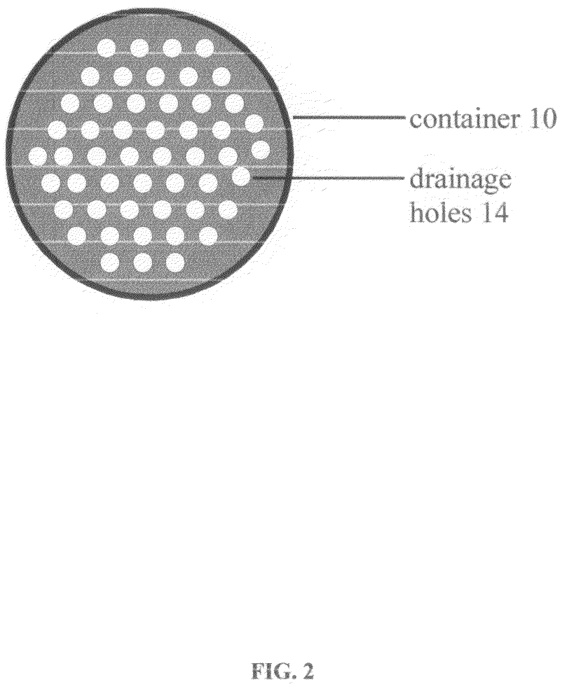

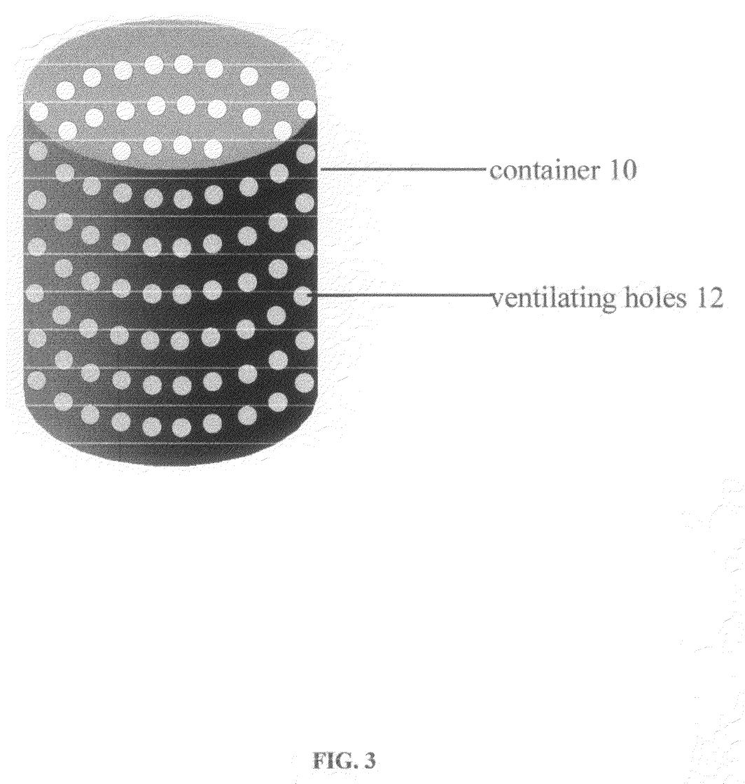

Referring to FIG. 1 to FIG. 3 a representative embodiment of the present invention is illustrated. In FIG. 1 there is shown a front view of a representative embodiment of the present invention having a cylindrical container 10 and a series of ventilating holes 12. In FIG. 2 there is shown a top view of a representative embodiment of the present invention having a cylindrical container 10 and a series of drainage holes 14. In FIG. 3 there is shown a perspective view of a representative embodiment of the present invention having a cylindrical container 10 and a series of ventilating holes 12.

In more detail, still referring to the representative embodiment of the present invention of FIG. 1 to FIG. 3, in FIG. 1 to FIG. 3 the container 10 is configured with a number of ventilating holes 12 to promote air circulation. The ventilating holes 12 of the container 10 of the present invention enable, promote and accelerate air circulation to evaporate and dry the organic payload thereby preventing the build-up of dangerous heat and the likelihood of spontaneous combustion while preventing the accumulation of objectionable fumes. The evaporation of liquid from the organic payload spawned by the present invention results in a significant automatic aggregate weight reduction for the container 10 and the payload as well. The drainage holes 14 shown in FIG. 2 provide outlet and relief to prevent the accumulation of standing water further aiding the drying and evaporation process. By themselves, the ventilating holes 12 of FIG. 1 and FIG. 3 and the drainage holes 14 of FIG. 2 effect a significant intrinsic weight reduction for the container 10 of FIG. 1 to FIG. 3 without significantly reducing strength. These additional benefits of the present invention ease the burden of lifting and transporting the waste for both the property owner and collector.

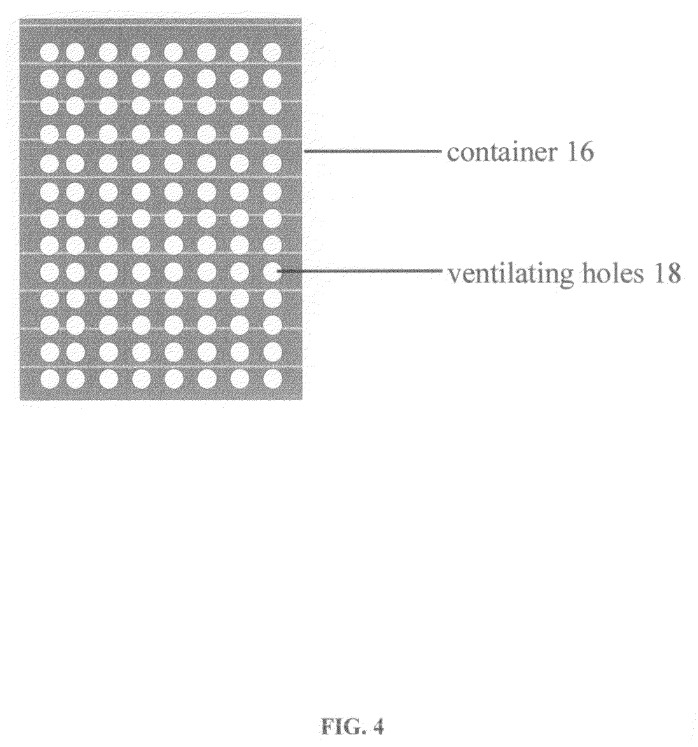

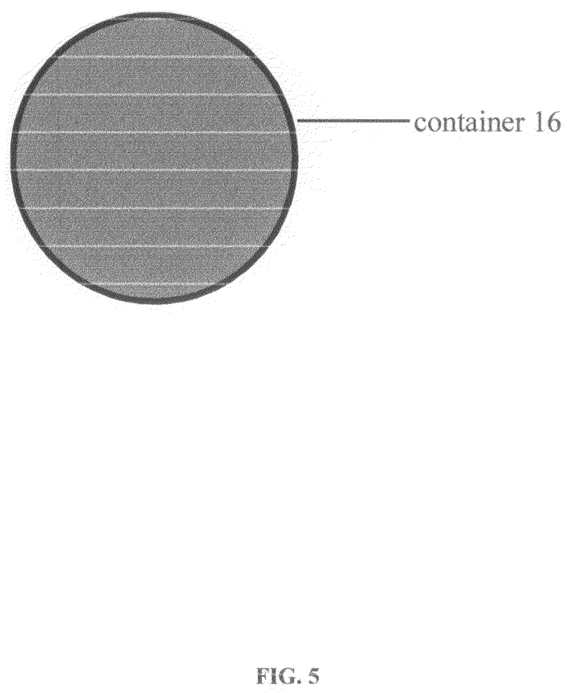

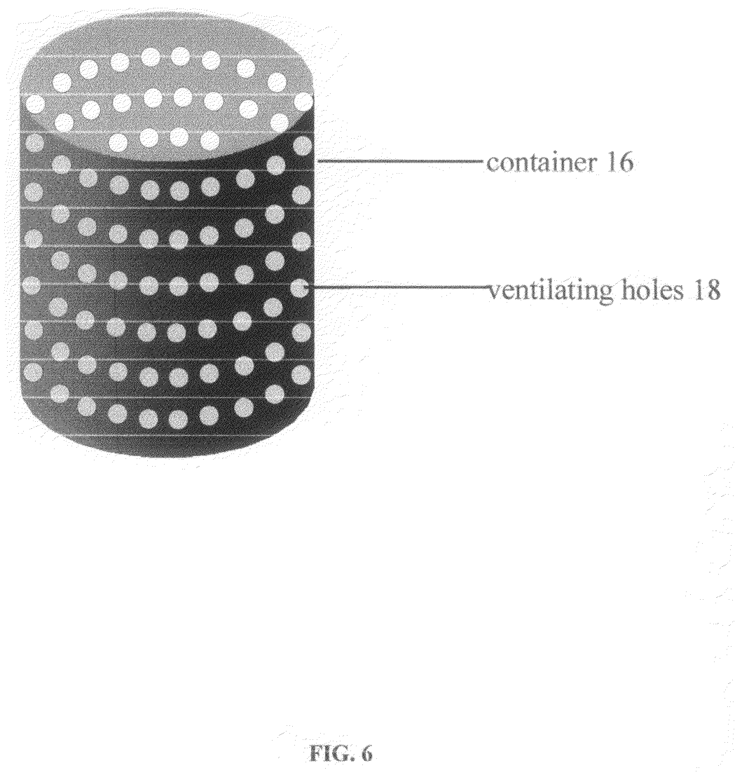

Referring now to FIG. 4 to FIG. 6 a first alternate embodiment of the present invention is illustrated. In FIG. 4 there is shown a front view of a first alternate embodiment of the present invention having a cylindrical container 16 and a series of ventilating holes 18. In FIG. 5 there is shown a top view of a first alternate embodiment of the present invention having a cylindrical container 16. In FIG. 6 there is shown a perspective view of a first alternate embodiment of the present invention having a cylindrical container 16 and a series of ventilating holes 18. In this first alternate embodiment of the present invention no drainage holes are provided in the container 16.

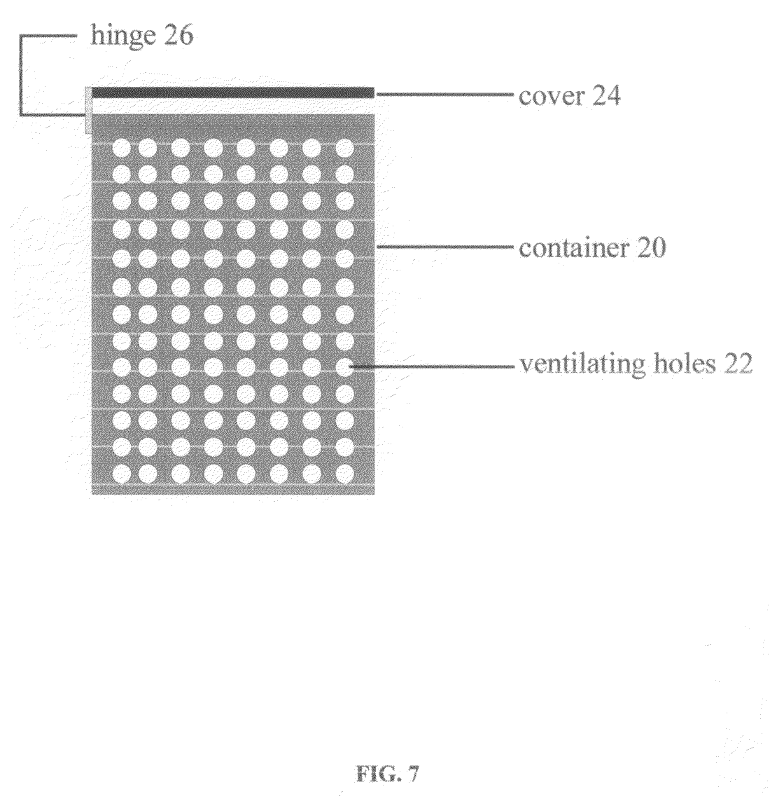

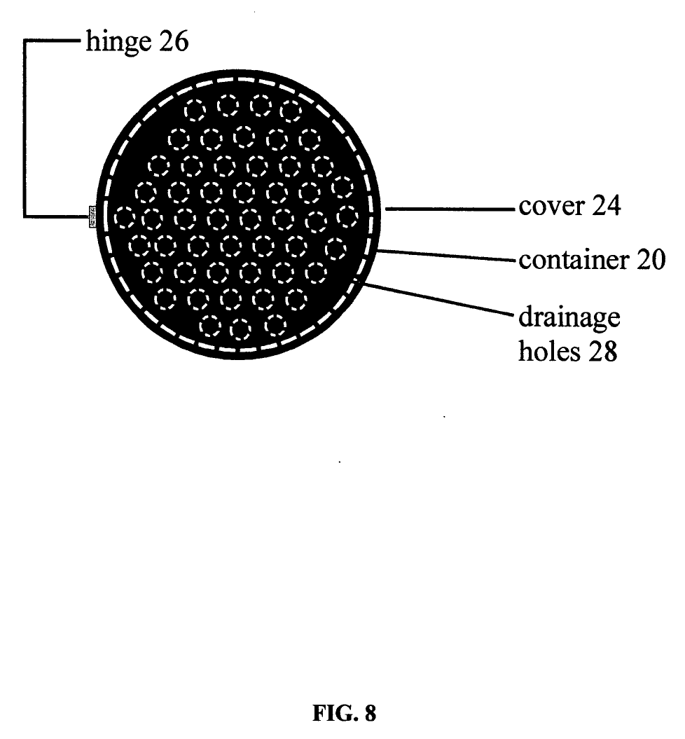

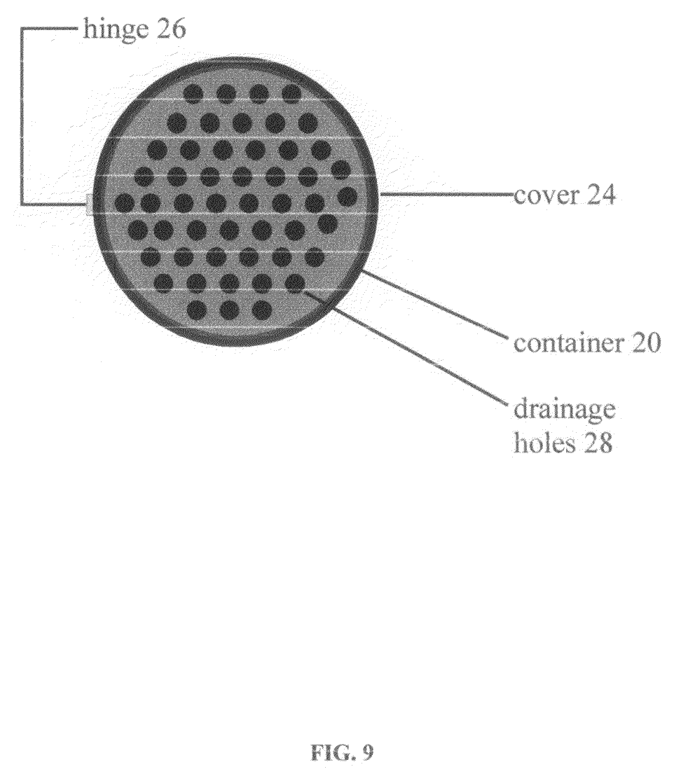

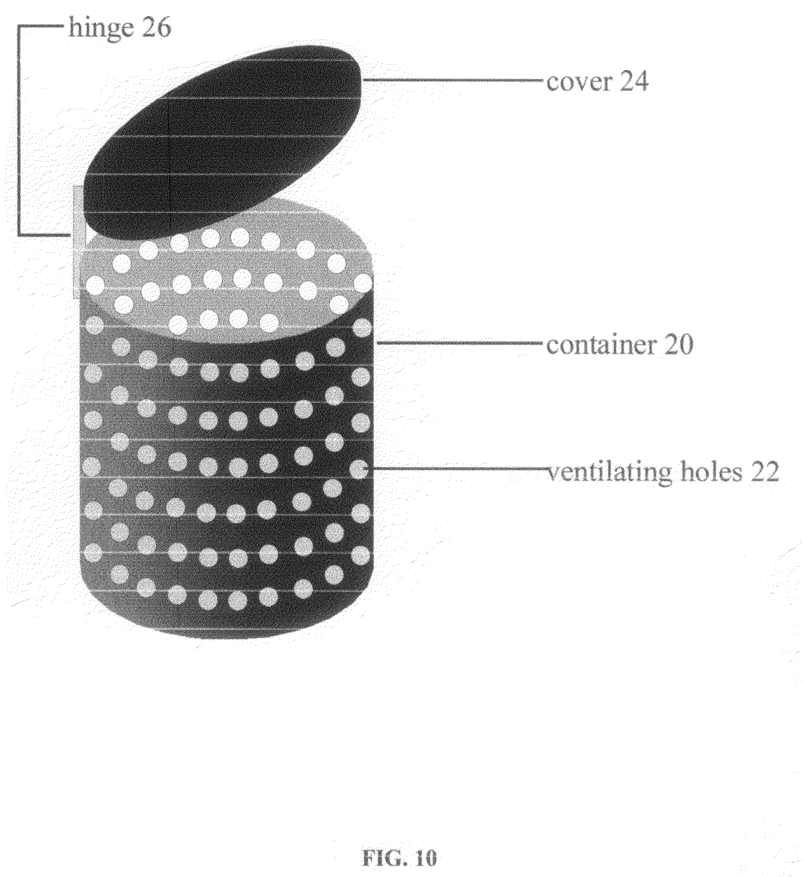

Referring now to FIG. 7 to FIG. 10 a second alternate embodiment of the present invention is illustrated. In FIG. 7 there is shown a front view of a second alternate embodiment of the present invention having a cylindrical container 20, a series of ventilating holes 22, a rotatable cover 24, and a hinge 26. In FIG. 8 there is shown a top view of a second alternate embodiment of the present invention having a cylindrical container 20, a rotatable cover 24, a hinge 26, and a series of drainage holes 28. In FIG. 9 there is shown a bottom view of a second alternate embodiment of the present invention having a cylindrical container 20, a rotatable cover 24, a hinge 26, and a series of drainage holes 28. In FIG. 10 there is shown a perspective view of a second alternate embodiment of the present invention having a cylindrical container 20, a series of ventilating holes 22, a rotatable cover 24, and a hinge 26. In this embodiment the rotatable cover 24 is rotatable by means of the hinge 26. When rotated to the down position the rotatable cover 24 rests over but above the container 20 providing additional protection from moisture by shielding the payload from precipitation while preserving the circulation conduit above the payload.

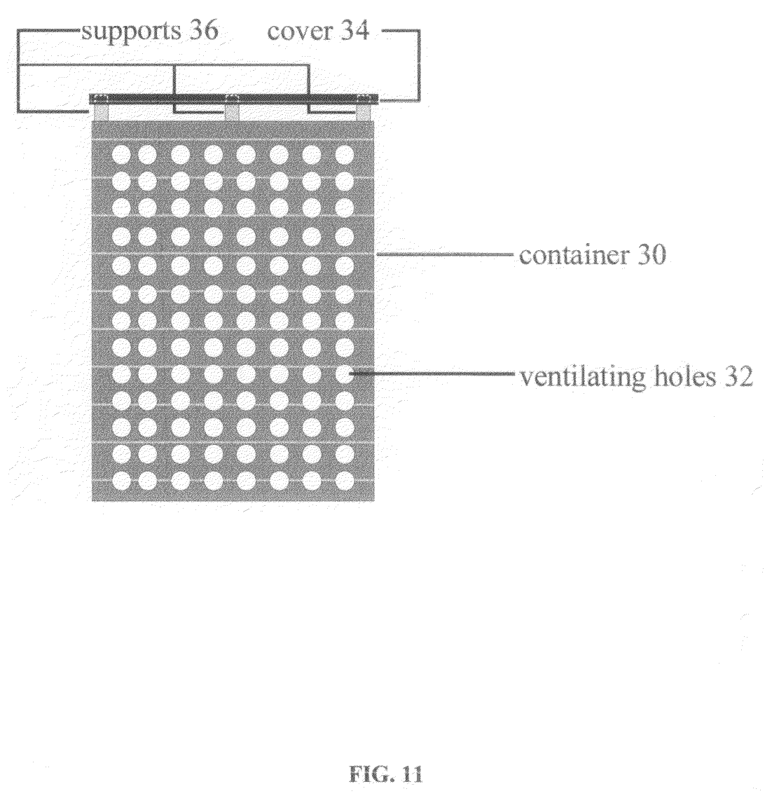

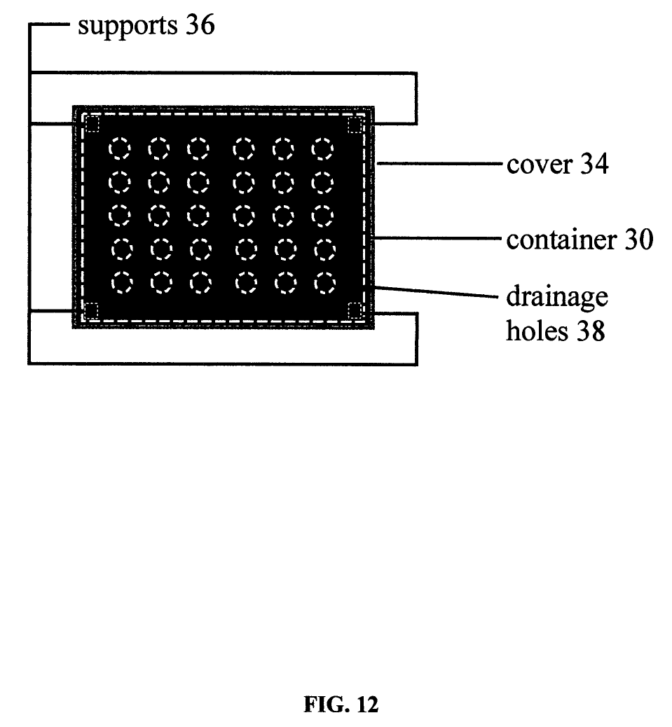

Referring now to FIG. 11 to FIG. 12 a third alternate embodiment of the present invention is illustrated. In FIG. 11 there is shown a front view of a third alternate embodiment of the present invention having a rectangular container 30, a series of ventilating holes 32, a removable cover 34, and supports 36. In FIG. 12 there is shown a top view of a third alternate embodiment of the present invention having a rectangular container 30, a removable cover 34, supports 36, and a series of drainage holes 38. When placed on the supports 36 the removable cover 34 rests over but above the container 30 providing additional protection from moisture by shielding the payload from precipitation while preserving the circulation conduit above the payload.

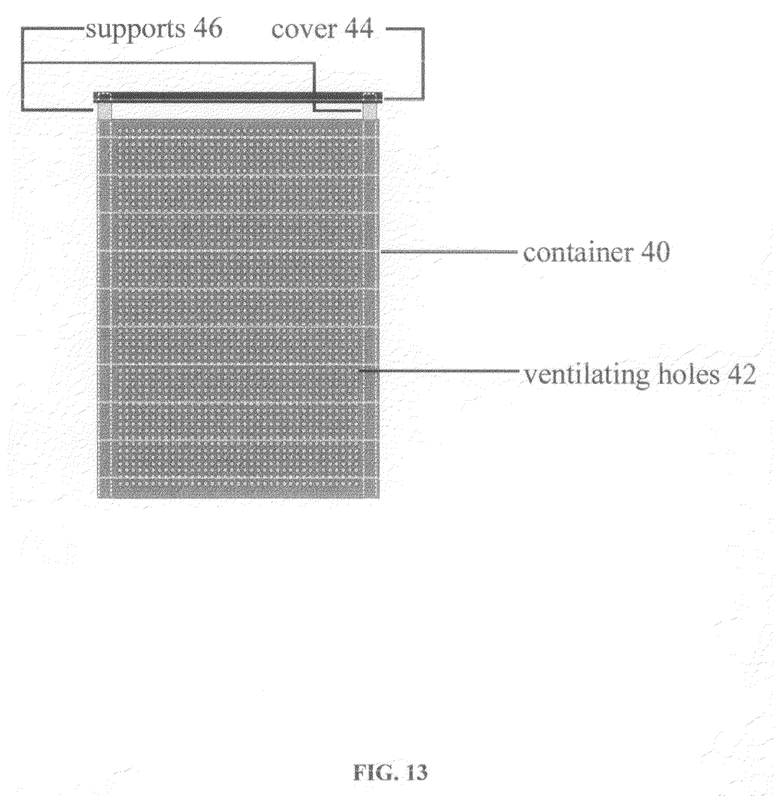

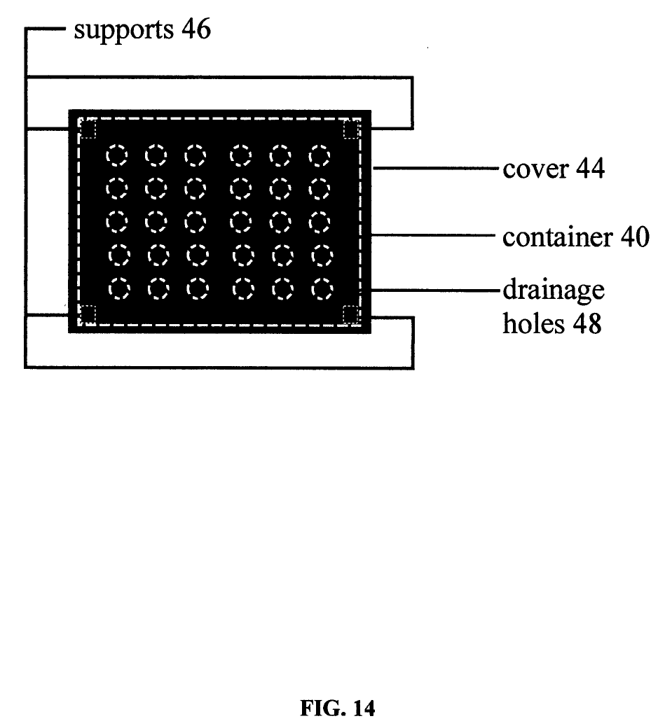

Referring now to FIG. 13 and FIG. 14 a fourth alternate embodiment of the present invention is illustrated. In FIG. 13 there is shown a front view of a fourth alternate embodiment of the present invention having a rectangular container 40, a series of ventilating holes 42, a removable cover 44, and supports 46. In FIG. 14 there is shown a top view of a fourth alternate embodiment of the present invention having a rectangular container 40, a removable cover 44, supports 46, and a series of drainage holes 48. When placed on the supports 46 the removable cover 44 rests over but above the container 40 providing additional protection from moisture by shielding the payload from precipitation while preserving the circulation conduit above the payload. In this fourth alternate embodiment of the present invention a mesh of fine wires spaced apart vertically and horizontally provide the ventilating holes 42 as illustrated in FIG. 13.

The construction details of the present invention are that the material for container 10 in the representative embodiment of the present invention shown in FIG. 1 to FIG. 3 and for container 16 in the first alternate embodiment of the present invention shown in FIG. 4 to FIG. 6, and for container 20 in the second alternate embodiment of the present invention shown in FIG. 7 to FIG. 10, and for container 30 in the third alternate embodiment of the present invention shown in FIG. 11 to FIG. 12, and for container 40 in the fourth alternate embodiment of the present invention shown in FIG. 13 to FIG. 14 be made of sufficient strength to accommodate practical measures of payload matter and may be constituted from, but are not limited to, any of the following:

Plastic

Metal

Cloth

Paper

Cardboard

Ceramic

Wood

The size, shape, and pattern of the of the container 10 in the representative embodiment of the present invention shown in FIG. 1 to FIG. 3 and of the container 16 in the first alternate embodiment of the present invention shown in FIG. 4 to FIG. 6, and of the container 20 in the second alternate embodiment of the present invention shown in FIG. 7 to FIG. 10, and of the container 30 in the third alternate embodiment of the present invention shown in FIG. 11 to FIG. 12, and of the container 40 in the fourth alternate embodiment of the present invention shown in FIG. 13 to FIG. 14 are infinitely variable.

The size, shape, and pattern of the of the ventilating holes 12 in the representative embodiment of the present invention shown in FIG. 1 and FIG. 3 and of the ventilating holes 18 in the first alternate embodiment of the present invention shown in FIG. 4 and FIG. 6, and of the ventilating holes 22 in the second alternate embodiment of the present invention shown in FIG. 7 and FIG. 10, and of the ventilating holes 32 in the third alternate embodiment of the present invention shown in FIG. 11, and of the ventilating holes 42 in the fourth alternate embodiment of the present invention shown in FIG. 13 are infinitely variable.

The size, shape, and pattern of the of the drainage holes 14 in the representative embodiment of the present invention shown in FIG. 2 and of the drainage holes 28 in the second alternate embodiment of the present invention shown in FIG. 8 and FIG. 9, and of the drainage holes 38 in the third alternate embodiment of the present invention shown in FIG. 12, and of the drainage holes 48 in the fourth alternate embodiment of the present invention shown in FIG. 14 are infinitely variable.

Although a few preferred embodiments have been shown and described, it will be appreciated by those skilled in the art that various changes and modifications might be made without departing from the scope of the invention. The terms and expressions used in the preceding specification have been used herein as terms of description and not of limitation, and there is no intention in the use of such terms and expressions of excluding equivalents of the features shown and described or portions thereof.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiments, methods, and examples, but by all embodiments and methods within the scope and spirit of the invention as claimed.

### ADVANTAGES OF THE PRESENT INVENTION

The advantages of the present invention include, without limitation, the alleviation of fire hazards and spontaneous combustion and foul odors from container payloads of decomposing organic material. The container of the invention is configured with multiple holes facilitating air circulation and drainage to promote natural drying, heat dispersal, decay retardation, and to impede and diffuse unsavory and objectionable aromas emanating from the payload matter. A rotatable or removable cover of the invention prevents water entry from rain while augmenting and sustaining airflow. By this method and apparatus the build-up of dangerous heat and likelihood of spontaneous combustion and the accumulation of objectionable fumes is greatly diminished. The ventilating holes of the container of the present invention enable and promote evaporation and drying of the organic payload providing significant and automatic weight reduction. Furthermore, the ventilating holes themselves constitute a significant container weight reduction without significantly reducing strength. These additional benefits of the present invention ease the burden of lifting and transporting the waste for both the property owner and collector.

1. An Aerating Organic Matter Container comprising a container with orifices for aerating organic waste material and water drainage.

2. The Aerating Organic Matter Container of claim 1 wherein odors produced by decaying yard waste payload are continuously ventilated via the aerating orifices to prevent the build-up of objectionable odors.

3. The Aerating Organic Matter Container of claim 1 wherein heat developed by the decaying yard waste payload is continuously ventilated via the aerating orifices to prevent spontaneous combustion.

4. The Aerating Organic Matter Container of claim 1 wherein water contained in the stored yard waste payload is continuously exhausted via the draining orifices to prevent the build-up of objectionable odors.

5. The Aerating Organic Matter Container of claim 1 wherein water contained in the stored yard waste payload is continuously exhausted via the draining orifices to reduce the aggregate weight of the container and yard waste payload.

6. The Aerating Organic Matter Container of claim 1 wherein water contained in the stored yard waste payload is continuously exhausted via the draining orifices to prevent heat build-up and the attendant likelihood of spontaneous combustion.
